(19) United States
(12) Patent Application Publication
(10) Pub. No.: US 2026/0099732 A1
(43) Pub. Date: Apr. 9, 2026

(54) Creating Semantic Knowledge Relationships for Electronic Health Record Systems Content

(71) Applicant: Oracle International Corporation, Redwood Shores, CA (US)

(72) Inventors: Natalee Agassi, Blue Bell, PA (US); Amitabh Saikia, Mountain View, CA (US); Vadim Khotilovich, Leawood, KS (US); Raefer Christopher Gabriel, San Mateo, CA (US); Jigar Shirish Patel, Lenexa, KS (US); Suhas Srinivas Uliyar, Woodinville, WA (US)

(73) Assignee: Oracle International Corporation, Redwood Shores, CA (US)

(21) Appl. No.: 19/299,926

(22) Filed: Aug. 14, 2025

Related U.S. Application Data
(60) Provisional application No. 63/705,426, filed on Oct. 9, 2024.

(51) Int. Cl. G06N 5/025; G16H 10/60; G16H 20/10; G16H 70/40

(52) U.S. Cl. CPC G06N 5/025; G16H 10/60; G16H 20/10; G16H 70/40

(57) ABSTRACT

A system and method for enhancing electronic health record (EHR) systems through integration of proprietary and standardized medical terminologies. Embodiments proprietary terminology concepts from electronic sources and identifies matching standardized medical terminology concepts. A knowledge graph is generated, incorporating both proprietary and standardized concepts along with their relationships. The system receives patient data, including problem lists, medication lists, and lab results. This data is analyzed against the knowledge graph to generate suggestions for the EHR system. These suggestions are then provided to the EHR system for display, enhancing clinical decision support and improving patient care.

### INCORPORATION BY REFERENCE; DISCLAIMER

The following application is hereby incorporated by reference application no. 63/705,426 filed on Oct. 9, 2025 entitled “Creating Semantic Knowledge Relationships For Electronic Health Record Systems Content”. The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to electronic health record (EHR) systems.

### BACKGROUND

Electronic health record (EHR) systems serve as digital repositories for patient health information. EHR systems store and manage a wide range of patient data, including medical histories, diagnoses, medications, treatment plans, immunization dates, allergies, radiology images, and laboratory test results. The systems provide real-time, patient-centered records that make information available instantly and securely to authorized users. EHR systems implement security measures to protect sensitive patient data, including encryption, access controls, and audit trails. The systems support interoperability standards, enabling seamless data exchange between different healthcare providers and organizations.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system for creating augmented knowledge relationships for EHR systems content in accordance with one or more embodiments;

FIG. 2 illustrates an example set of operations for creating augmented knowledge relationships for EHR systems content in accordance with one or more embodiments;

FIG. 3 illustrates an example operational workflow to produce clinical decision support recommendations within electronic health record systems; and

FIG. 4 shows a block diagram that illustrates a system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. AUGMENTED KNOWLEDGE RELATIONSHIP ARCHITECTURE
3. CREATING AUGMENTED KNOWLEDGE RELATIONSHIPS FOR ELECTRONIC HEALTH RECORD SYSTEMS
4. PATIENT MEDICATION ANALYSIS EXAMPLE
5. ADDITIONAL INFORMATION
6. PRACTICAL APPLICATIONS, ADVANTAGES & IMPROVEMENTS
7. MISCELLANEOUS; EXTENSIONS
8. HARDWARE OVERVIEW

### 1. General Overview

One or more embodiments process proprietary terminology concepts to enhance medical data interoperability. Data interoperability refers to the ability of different information systems, applications, and devices to share and use data seamlessly, which provides numerous technical benefits as described further herein. The processing for enhancing data interoperability includes identifying matching standardized medical terminology concepts from a standardized medical terminology source. Natural language processing (NLP) techniques analyze the proprietary concepts to extract meaningful information. A “concept” refers to a digital representation of medical or clinical information, such as diseases, findings, procedures, drug treatments, and anatomical structures. A concept may serve as a building block that allows a database to organize and link diverse medical information through a structured, hierarchical approach, enabling efficient retrieval and use of this information in applications like clinical decision support system (CDSS) software. Proprietary terminology concepts are representations that include non-standardized medical/clinical terminology and/or non-standardized data structures such as proprietary database schemas and object relationships. Standardized medical concepts provide a common language and structure for representing healthcare data. In one example, the standardized medical terminology source is Systemized Nomenclature of Medicine—Clinical Terms (SNOMED CT), a comprehensive clinical healthcare terminology, and the proprietary terminology includes medicine information, such as Multum, which employs a proprietary system for organizing drug information, including its own hierarchical classification system.

One or more embodiments generate a knowledge graph incorporating diverse medical terminologies. The knowledge graph integrates proprietary terminology concepts, standardized medical terminology concepts, and relationships between them. Relationships between medications and conditions are established by linking drug indications and side effects to corresponding diagnoses and problems within the knowledge graph. The resulting structure enables complex queries across different medical knowledge domains.

One or more embodiments analyze patient data against the knowledge graph to generate clinical suggestions. The analysis includes processing problem lists, medication lists, and lab results. The generated suggestions are then provided to an electronic health record (EHR) system for display to healthcare providers.

One or more embodiments process natural language queries about patient medications and conditions. The knowledge graph serves as the foundation for interpreting and responding to these queries. A chatbot interface receives queries from the EHR system, allowing for intuitive interactions. In some cases, the chatbot interface accepts auditory input, that is converted into text queries for processing.

One or more embodiments described in this Specification and/or recited in the claims may not be included in this General Overview section.

### 2. Augmented Knowledge Relationship Architecture

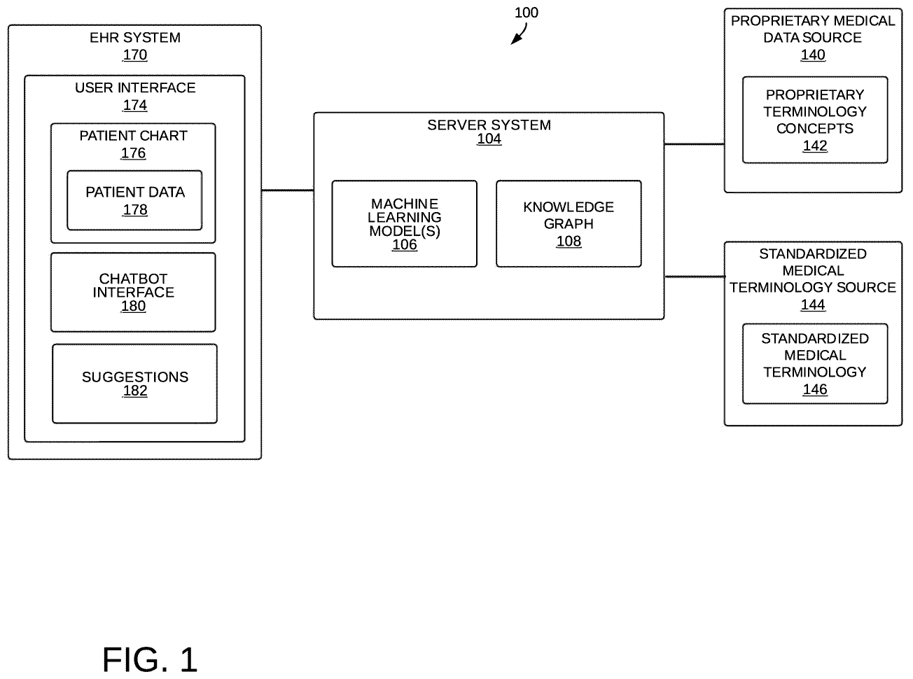

FIG. 1 illustrates a system for creating augmented knowledge relationships for EHR systems content in accordance with one or more embodiments. System 100 summarizes proprietary medical terminology to create a knowledge graph integrating standardized medical concepts, analyzes patient data against this graph, and generates clinical suggestions for electronic health record systems, enhancing interoperability and decision support in healthcare.

As illustrated in FIG. 1, system 100 comprises server system 104, machine learning model(s) 106, knowledge graph 108, proprietary medical data source 140, proprietary terminology concepts 142, standardized medical terminology source 144, standardized medical terminology 146, EHR system 170, user interface 174, patient chart 176, patient data 178, chatbot interface 180, and suggestions 182. In one or more embodiments, system 100 may include more components or fewer components than the components illustrated in FIG. 1.

In an embodiment, EHR system 170 serves as the central hub for managing patient health records in a healthcare environment. EHR system 170 stores and organizes patient information, including detailed medical histories, current and past diagnoses, prescribed medications, treatment plans, and outcomes of previous interventions. EHR system 170 supports interoperability standards, enabling seamless data exchange with other healthcare systems and devices.

In an embodiment, user interface 174 is the gateway through which healthcare professionals interact with patient data. User interface 174 efficiently organizes, displays, and facilitates the input of medical information. User interface 174 typically includes features like patient dashboards, data entry forms, search functions, and alert systems. User interface 174 enables doctors and nurses to easily access and update patient records, order tests, review results, and review documents.

In an embodiment, patient chart 176 within EHR system 170 organizes and displays patient-specific information in a comprehensive and easily navigable format. Patient chart 176 serves as a centralized hub for relevant patient data, including medical history, current medications, allergies, lab results, and treatment plans. Patient chart 176 presents patient information in a logical, hierarchical structure, allowing healthcare providers to quickly access and update specific sections as needed. Interactive elements such as tabs, dropdown menus, and clickable icons facilitate efficient navigation through the various components of the patient's record, ensuring critical information is readily available to support informed clinical decision-making.

In an embodiment, patient data 178 encompasses relevant medical information for individual patients, forming a comprehensive digital health record. Patient data 178 includes detailed medical information such as a comprehensive medical history, including past diagnoses, surgeries, and treatments, current and past medications, including dosages and adverse reactions, allergies and sensitivities, immunization records, lab and imaging results, vital signs and other biometric data, treatment plans and care goals, and notes from healthcare provider encounters.

In an embodiment, chatbot interface 180 provides an AI-driven interface within the EHR system 170. Chatbot interface 180 allows healthcare professionals to interact with the system using natural language queries and commands. Chatbot interface 180 is an interface for various tasks such as retrieving specific patient information, scheduling appointments, ordering tests, and providing basic clinical decision support. Chatbot interface 180 uses NLP to interpret user inputs and interacts with machine learning algorithms to provide responses. Chatbot interface 180 interacts with server system 104 to obtain information from machine learning model(s) 106 and knowledge graph 108. Chatbot interface 180 sends queries to machine learning model(s) 106. In one example, the queries are converted from audio data.

A medical terminology is a vocabulary for clinical documentation and reporting. Medical terminology systems provide a common language for healthcare professionals to describe diagnoses, symptoms, procedures, and other health-related concepts. Medical terminology systems aim to reduce ambiguity and improve communication across different healthcare settings and information systems.

In an embodiment, standardized medical terminology source 144 incorporates internationally recognized coding systems such as SNOMED CT, the International Classification of Diseases, 10th Revision (ICD-10), and Logical Observation Identifiers, Names, and Codes (LOINC). Standardized medical terminology source 144 undergoes regular updates to reflect the latest advancements in medical knowledge and practices. The structured hierarchy within standardized medical terminology source 144 enables precise and unambiguous representation of medical concepts.

In an embodiment, standardized medical terminology 146 forms the backbone of system-wide communication. Standardized medical terminology 146 enables seamless data exchange between different healthcare systems and facilitates accurate billing and reimbursement processes. The use of standardized medical terminology 146 supports clinical decision support systems by providing a consistent language for defining medical conditions, treatments, and outcomes.

In an embodiment, proprietary medical data source 140 encompasses datasets owned by healthcare organizations, pharmaceutical companies or other third-party organizations. In one example, proprietary terminology concepts 142 is a drug information database, such as Multum. Multum is a comprehensive drug information database and clinical decision support system widely used in the healthcare industry that includes drug interactions, allergy alerts, dosing guidelines, side effect information, and generic and brand name listings. Much of such drug information databases is not immediately interoperable with standardized medical terminology 146.

In an embodiment, server system 104 generates knowledge graph 108 from the proprietary terminology concepts 142 and the standardized medical terminology 146 using machine learning model(s) 106. In one example, proprietary terminology concepts 142 undergo a extraction process. Natural language processing algorithms parse through unstructured text from proprietary medical data source 140 to identify and categorize relevant medical terms and concepts. Proprietary terminology concepts 142 undergo a normalization process to align with standardized terminologies while preserving their unique attributes.

In an embodiment, machine learning model(s) 106 encompass one or more algorithms, such as large language models (LLMs) tailored for medical data analysis. Machine learning model(s) 106 utilize different techniques, such as deep learning, NLP, and statistical analysis to extract meaningful patterns from complex medical datasets. The adaptive nature of machine learning model(s) 106 enables them to refine their predictive capabilities over time, leading to increasingly accurate insights for healthcare providers.

In an embodiment, a LLM is a type of artificial intelligence (AI) system designed to understand, generate, and manipulate human language at a large scale. These models are trained on vast amounts of text data, allowing them to recognize patterns, context, and nuances in language. LLMs perform a wide range of tasks, including text generation, translation, summarization, question-answering, and even basic reasoning. They use complex neural network architectures, typically based on transformer models, to process and generate text. In one example, an LLM undergoes training and fine-tuning using domain-specific medical corpora to improve its understanding of specialized medical terminology and concepts. In one example, an LLM incorporates attention mechanisms and transformer architectures to capture long-range dependencies in medical texts.

In an embodiment, knowledge graph 108 represents medical concepts and their interconnections in a structured format. Knowledge graph 108 incorporates the proprietary terminology concepts, the standardized medical terminology, and relationships between the proprietary terminology concepts and the standardized medical terminology.

In an embodiment, server system 104 with machine learning model(s) 106 and knowledge graph 108 generates suggestions 182. Suggestions 182 take into account patient-specific factors, such as comorbidities and medication interactions, to provide personalized recommendations. In one example, the system assigns confidence scores to suggestions 182, allowing healthcare providers to quickly assess the reliability of recommendations. In one example, suggestions 182 are accompanied by explanations of the underlying reasoning, promoting transparency and trust in the system's decision-making process.

In one or more embodiments server system 104, machine learning model(s) 106, knowledge graph 108, proprietary medical data source 140, proprietary terminology concepts 142, standardized medical terminology source 144, standardized medical terminology 146, EHR system 170, user interface 174, patient chart 176, and chatbot interface 180 refer to hardware and/or software configured to perform operations described herein for information storage and retrieval.

In an embodiment, server system 104, machine learning model(s) 106, knowledge graph 108, proprietary medical data source 140, proprietary terminology concepts 142, standardized medical terminology source 144, standardized medical terminology 146, EHR system 170, user interface 174, patient chart 176, and chatbot interface 180 are implemented and/or stored on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

### 3. Creating Augmented Knowledge Relationships for Electronic Health Record Systems

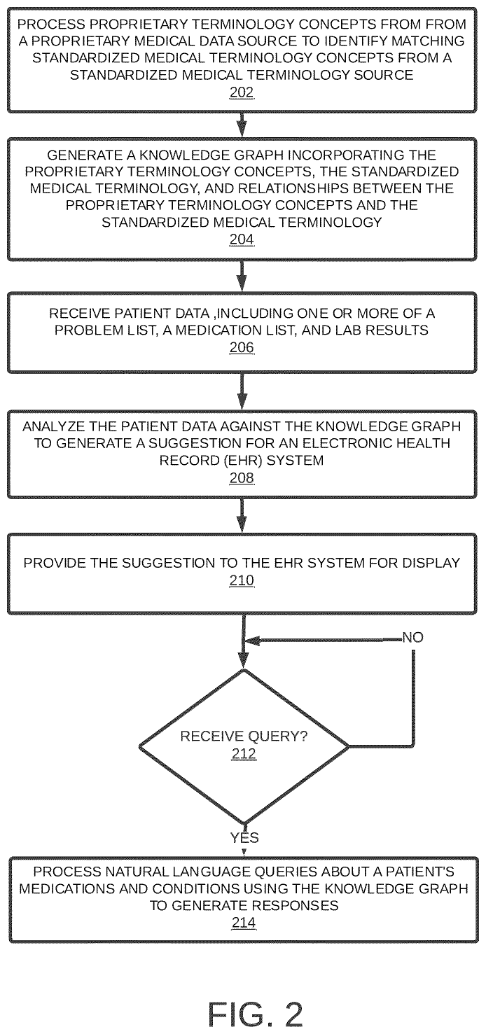

FIG. 2 illustrates an example set of operations for creating augmented knowledge relationships for EHR systems content in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

In an embodiment, the system processes proprietary terminology concepts from a proprietary medical data source to identify matching standardized medical terminology concepts from a standardized medical terminology source (Operation 202). The system analyzes the proprietary concepts using NLP techniques to find corresponding standardized terms. The matching process enables integration of proprietary and standardized medical vocabularies.

In an embodiment, the system employs natural language processing techniques to identify corresponding terms. The proprietary terminology concepts and standardized medical terminology concepts are tokenized and converted into vector representations. Semantic similarity matching calculates vector representations of proprietary terms and compares these representations against standardized medical terminology concepts. In one example, the system calculates cosine similarity measurements between vector representations of proprietary terminology concepts and standardized medical terminology concepts. The system calculates the cosine of the angle between feature vectors for the proprietary terminology concepts and standardized medical terminology concepts. Similarity scores close to one indicate strong semantic correspondence between proprietary terminology concepts and standardized medical terminology concepts.

In an embodiment, the system generates a knowledge graph incorporating the proprietary terminology concepts, the standardized medical terminology, and relationships between the proprietary terminology concepts and the standardized medical terminology (Operation 204). The knowledge graph represents medical concepts and their interconnections in a structured format. Relationships between proprietary and standardized terms are explicitly modeled within the graph structure.

In an embodiment, the system receives patient data that include one or more of a problem list, a medication list, and lab results (Operation 206). The patient data originates from EHR systems or other clinical data sources. The received data includes information about a patient's medical conditions, prescribed medications, and diagnostic test results.

In an embodiment, the system analyzes the patient data against the knowledge graph to generate a suggestion for EHR system (Operation 208). The analysis involves traversing the knowledge graph to find relevant connections between the patient's data and medical knowledge represented in the graph. Suggestions generated from the analysis provide clinically relevant insights or recommendations.

In an embodiment, the system provides the suggestion to the EHR system for display (Operation 210). The suggestions are formatted and transmitted to the EHR system through an appropriate interface. EHR users view the suggestions within their workflow to inform clinical decision-making.

In an embodiment, the system checks for the presence of a query (Operation 212). The query originates from user input or automated processes within the EHR system. Queries represent requests for information or analysis related to patient care. If there is a query, the system proceeds to operation 214. If there is no query, the system repeats operation 212.

In an embodiment, the system processes natural language queries about a patient's medications and conditions using the knowledge graph to generate responses when a query is received (Operation 214). Natural language processing techniques interpret the query intent. The knowledge graph is traversed to find relevant information. Responses are formulated based on the graph analysis results.

In an embodiment, the system ontologically represents proprietary terminology data from sources such as Multum. The system performs NLP on proprietary terminologies to identify matching standardized medical terminology concepts from sources like SNOMED CT. The system leverages semantic features of SNOMED CT to enhance accuracy and relevance of search results.

In an embodiment, the system employs ML algorithms to analyze patient data including medical histories, lab results, imaging studies, and clinical notes. The system identifies patterns and correlations indicative of medical problems, conditions, and diagnoses. The system incorporates NLP to extract meaningful information from unstructured clinical notes and documentation. The system extracts medical concepts within specific time frames related to documented problems in EHR. The system processes and matches extracted concepts to standard terminologies.

In an embodiment, the system uses data analysis to propose new classes or properties to add to a knowledge base. The system enriches the semantic structure of the knowledge base to improve search capabilities. The system creates and enhances semantic knowledge relationships with content from EHR systems, proprietary terminology, and standardized medical terminologies like SNOMED CT. The system facilitates voice-activated and active search functionalities. The system improves accuracy and efficiency of retrieving relevant patient information through enhanced semantic relationships.

In one embodiment, the system processes proprietary terminology from EHRs to create a standardized, computable knowledge base. The system extracts concepts from proprietary drug databases, like Multum, using NLP techniques. Extracted concepts are matched to standardized terminologies such as SNOMED CT. The system builds a graph representation that incorporates the proprietary concepts, the standardized codes, and the relationships between them. Relationships between medications and conditions are established by linking drug indications and side effects to corresponding diagnoses and problems. The resulting knowledge graph enables querying across proprietary and standardized terminologies. Queries traverse the graph to find connections between medications, conditions, side effects, and other clinical concepts.

In an embodiment, the system constructs the knowledge graph through a multi-stage process that begins with concept extraction from proprietary terminology databases such as Multum and establishes standardized mappings through automated matching algorithms that achieve correspondence thresholds, such as seventy-five percent or higher. The extraction process parses proprietary database schemas to identify medication information including drug classifications, therapeutic indications, contraindications, and side effect profiles, then maps these structural elements to corresponding ontological classes and properties within standardized terminologies like SNOMED CT. Matching occurs through natural language processing methods that break down textual descriptions into constituent words, apply word rooting algorithms to identify stemmed forms, and calculate similarity measurements between proprietary term representations and standardized concept descriptors. The system creates graph nodes for each identified concept such as by using Resource Description Framework triplet structures, establishing edges that represent specific relationships such as “hasMultumIndication,” “hasMultumCommonSideEffect,” and “hasContraindication” between medication concepts and condition concepts.

In one embodiment, the system provides an application programming interface (API) for uploading patient data, such as problem lists, medication lists, and lab results. Uploaded data is analyzed against the knowledge graph to generate insights. Natural language queries about a patient's medications and conditions are processed using the graph. Results are passed through an LLM to generate concise, human-readable responses.

Dosing information may be challenging to fully codify due to complexity and variability, in one embodiment, the system uses large language models to interpret and reason about unstructured dosing text. This allows for evaluating if a patient is on optimal medication doses for their conditions. The knowledge graph enables linking problems in the patient record to potential medication side effects or indications.

In an embodiment, the system utilizes large language models to interpret unstructured dosing text by processing medication instructions that cannot be easily codified through traditional structured approaches. The large language model receives dosing text as input and applies transformer-based neural network architectures to analyze contextual relationships between words and phrases within medication instructions. Processing occurs through attention mechanisms that identify key dosing parameters such as frequency indicators, quantity specifications, timing constraints, and conditional instructions within unstructured text. The model generates structured output by extracting dosing components and converting free-text instructions into standardized formats that specify numerical doses, administration schedules, and duration parameters. Large language model processing deals with complex dosing scenarios including sliding scale protocols for medications like insulin and multi-day dose packs for steroids where instructions involve variable timing and quantities.

In one embodiment, the system improves interoperability by connecting proprietary terminologies used within EHRs to standardized terminologies used for health information exchange. Standardization allows patient data to be more easily transferred between healthcare organizations. The knowledge graph provides a richer representation of clinical concepts compared to typical terminology value sets used in health IT systems.

In one embodiment, the system enables more advanced clinical decision support by providing a comprehensive view of medication-condition relationships. When prescribing medications, the system identifies potential interactions or contraindications based on a patient's full medical history. Side effect profiles are linked to specific medications and conditions, allowing for more targeted monitoring.

In an embodiment, the system identifies potential interactions and contraindications by traversing knowledge graph relationships to detect medication-condition conflicts and cross-referencing patient medical histories against contraindication databases. The identification process queries the knowledge graph for paths connecting patient medications to documented contraindications, following relationship edges that link drug concepts to condition concepts marked as incompatible or requiring special monitoring. Graph traversal algorithms explore multi-hop connections between medications and conditions, identifying both direct contraindications and indirect interactions that occur through shared metabolic pathways or overlapping therapeutic effects. The system maintains contraindication severity classifications, ranking identified conflicts based on clinical significance levels ranging from minor monitoring requirements to absolute contraindications requiring immediate medication discontinuation. Patient medical history analysis involves parsing structured problem lists and medication histories to identify documented conditions, allergies, and previous adverse drug reactions that inform contraindication checking.

In an embodiment, the system extracts dosing information from proprietary medical data sources where medication instructions appear as unstructured text rather than standardized data fields, then applies large language models to interpret complex dosing regimens that cannot be easily codified through conventional structured approaches. Dosing information extraction processes textual medication instructions that include variable timing protocols such as sliding scale dosing for insulin, multi-day dose packs for steroids like methylprednisolone, and antibiotic regimens such as Z-pack formulations where instructions specify taking two tablets on day one followed by one tablet daily for days two through four.

In one embodiment, graph queries support various use cases like identifying all medications prescribed for a particular condition or finding alternative treatments for patients with allergies. The system suggests medications that are missing from a patient's regimen based on their documented problems. By codifying information from drug monographs, the system makes previously inaccessible knowledge computable and actionable.

In one embodiment, NLP and ML techniques are employed to continuously refine and expand the knowledge graph. As new drug information becomes available, the system extracts relevant concepts and integrates them into the existing structure. Ontological representation allows for flexible querying at different levels of granularity to match the varying specificity of clinical documentation.

In an embodiment, the system employs natural language processing and machine learning techniques to continuously refine and expand the knowledge graph through automated concept extraction from new drug information sources and relationship validation algorithms. The refinement process monitors pharmaceutical databases, regulatory filings, and clinical literature for newly published drug information, applying document parsing algorithms to extract structured data from unstructured text sources. Concept extraction utilizes named entity recognition models trained on medical terminology to identify drug names, indication statements, contraindication descriptions, and adverse effect reports within source documents. The system applies concept normalization techniques that map extracted terms to existing knowledge graph entities or create new concept nodes when novel medications or conditions are identified. Relationship extraction employs dependency parsing and semantic role labeling to identify connections between medications and clinical concepts, establishing new graph edges when valid relationships are discovered. Machine learning models trained on existing knowledge graph patterns predict relationship likelihood and validate proposed connections against established medical knowledge. Integration workflows incorporate new concepts and relationships into the existing graph structure while maintaining semantic consistency and resolving potential conflicts through automated reasoning and human expert validation processes.

In one embodiment, voice-activated interfaces leverage the knowledge graph to support conversational interactions with clinicians. Providers ask questions about a patient's medications or conditions and receive synthesized responses. The system reduces cognitive burden on clinicians by quickly surfacing relevant information from complex patient records.

In one embodiment, deduplication of medication lists is enhanced by leveraging semantic understanding of drug information. The system distinguishes between loading doses and maintenance doses of the same medication, preserving important clinical distinctions. Intelligent merging of medication data from multiple sources improves the accuracy of reconciled medication lists. Deduplication further reduces system storage overhead costs and query execution times, improving system responsiveness.

In one embodiment, the system facilitates problem-oriented views of EHRs by linking medications, lab results, and other clinical data to specific conditions. Clinicians rapidly assess a patient's status for a given problem by viewing relevant information in context. The knowledge graph supports tracking of disease progression and treatment efficacy over time.

In one embodiment, automated summarization capabilities distill key information from lengthy patient records into concise overviews. The system extracts salient details about a patient's conditions, medications, and recent clinical events. Generated summaries help providers quickly orient themselves to a patient's history before encounters.

In one embodiment, pharmacogenomic data integration allows the system to provide personalized medication recommendations. Genetic markers associated with drug metabolism or efficacy are linked to relevant medications in the knowledge graph. The system flags potential issues or suggests dosing adjustments based on a patient's genetic profile.

In one embodiment, the system incorporates regulatory information and clinical guidelines into the knowledge graph, linking medications to approved indications and recommended usage protocols. Alerts are generated when prescribing practices deviate from established guidelines or when new safety warnings are issued for specific drugs.

### 4. Patient Medication Analysis Example

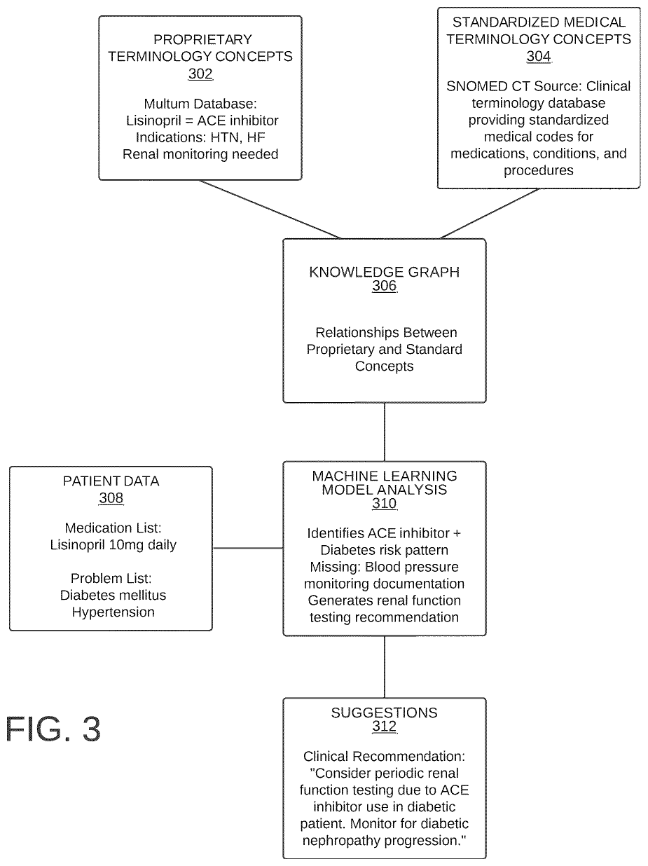

FIG. 3 illustrates an example operational workflow to produce clinical decision support recommendations within electronic health record systems. The example shown in in FIG. 3 should not be construed as limiting the scope of one or more embodiments.

In an embodiment, FIG. 3 is an example operational flow for creating augmented knowledge relationships for EHR systems content, demonstrating the integration and analysis of medical terminology data to generate clinical suggestions for a specific patient scenario involving diabetes management and cardiovascular medications.

In an embodiment, proprietary terminology concepts 302 comprise data extracted from proprietary medical data sources such as the Multum database containing comprehensive drug-specific information. Proprietary terminology concepts 302 include detailed medication profiles such as drug classifications, therapeutic indications, contraindications, and monitoring requirements. In the specific example, proprietary terminology concepts 302 classify Lisinopril as an ACE inhibitor medication with documented indications for hypertension and heart failure treatment, along with specific requirements for renal monitoring protocols. The proprietary data specifies that ACE inhibitors such as Lisinopril require periodic assessment of kidney function due to potential effects on renal blood flow and filtration rates.

In an embodiment, standardized medical terminology concepts 304 comprise data originating from standardized medical terminology sources such as SNOMED CT providing clinical terminology database functionality. Standardized medical terminology concepts 304 supply internationally recognized standardized medical codes and hierarchical classifications for medical concepts such as medications, conditions, procedures, and clinical findings. The standardized structure enables consistent medical documentation and data exchange across different healthcare systems and organizations, providing unified coding for conditions such as diabetes mellitus and hypertension.

In an embodiment, knowledge graph 306 comprises data incorporating proprietary terminology concepts 302, standardized medical terminology concepts 304, and defined relationships between the proprietary terminology concepts and the standardized medical terminology. Knowledge graph 306 creates a unified representation that bridges proprietary drug information with standardized clinical codes, establishing semantic connections that enable cross-referencing between different medical terminology systems. In the demonstrated example, knowledge graph 306 establishes relationships between ACE inhibitor medications and diabetic complications, linking Lisinopril therapy to diabetic nephropathy monitoring requirements and connecting hypertension management protocols with diabetes care guidelines.

In an embodiment, patient data 308 that includes one or more of a problem list, a medication list, and lab results received from EHR systems. Patient data 308 includes structured medical information such as current medications with dosages, documented medical conditions, and diagnostic test results. The specific example shows patient data 308 including a medication list specifying Lisinopril 10 mg administered daily and a problem list documenting two concurrent medical conditions: diabetes mellitus and hypertension. This combination represents a common clinical scenario where cardiovascular and endocrine conditions require coordinated management strategies.

In an embodiment, machine learning model analysis 310 processes patient data 308 against knowledge graph 306 to identify clinical patterns and generate targeted suggestions for an EHR system. Machine learning model analysis 310 traverses the relationships within knowledge graph 306 to detect clinically relevant associations such as the combination of ACE inhibitor therapy with diabetes risk factors. Machine learning model analysis 310 encodes patient information as feature vectors that represent medications as concept identifiers, problems as standardized terminology codes, and laboratory values as normalized numerical ranges. Graph embedding techniques create vector representations of knowledge graph concepts that capture semantic relationships between medications, conditions, and clinical findings, enabling mathematical similarity calculations between patient data elements and graph concepts. The machine learning model applies attention mechanisms to weight the importance of different patient data elements based on their relevance to specific clinical scenarios, focusing analysis on medication-condition pairs that demonstrate strong associative patterns within the knowledge graph. Pattern recognition algorithms identify clusters of related concepts in patient data that correspond to known clinical syndromes or medication interaction profiles represented in the knowledge graph structure. In the demonstrated example, machine learning model analysis 310 identifies the specific pattern of ACE inhibitor medication use in a diabetic patient, recognizes missing blood pressure monitoring documentation, and correlates this combination with established clinical guidelines requiring enhanced renal function surveillance. The model generates clinical suggestions by ranking potential recommendations based on confidence scores derived from knowledge graph relationship strengths, patient data similarity to training examples, and clinical guideline adherence metrics. Output generation combines machine learning predictions with rule-based clinical logic to produce actionable recommendations that include monitoring requirements, alternative treatment options, and risk mitigation strategies tailored to individual patient profiles. The analysis generates a renal function testing recommendation based on the increased risk of diabetic nephropathy progression in patients receiving ACE inhibitor therapy.

In an embodiment, suggestions 312 represent the clinical recommendations generated through ML model analysis 310 and provided to the EHR system for display to healthcare providers. Suggestions 312 deliver actionable clinical guidance such as monitoring recommendations, potential drug interactions, and preventive care protocols tailored to the specific patient profile. The specific example demonstrates suggestions 312 that provide targeted clinical guidance stating “Consider periodic renal function testing due to ACE inhibitor use in diabetic patient. Monitor for diabetic nephropathy progression” based on the comprehensive analysis of the patient's concurrent diabetes and hypertension conditions, Lisinopril medication regimen, and established clinical relationships within knowledge graph 306. This recommendation addresses the increased risk of kidney complications when ACE inhibitors are used in diabetic patients, providing healthcare providers with evidence-based guidance for optimal patient monitoring and care coordination.

### 5. Additional Information

In an embodiment, a reference enterprise data model establishes cross-enterprise processes for governance, cataloging entity assets, validation, enforcement, and team review activities. The reference enterprise data model delivers structured phases with defined deliverables and measurable values while identifying delegates from participating teams. Initial phases involve cataloging existing entities and attributes for individual domains, followed by creation of base reference enterprise data model structures that hold entities and attributes with semantic understanding mapped to data standards.

In an embodiment, the reference enterprise data model provides pre-coordinated and post-coordinated concepts and expressions that determine interoperability levels and semantic commonalities. The reference enterprise data model enables semantic explanation capabilities, identifies interoperability patterns and integration patterns, and supports automated building and testing processes. Base structures for business subdomain entity data models encompass finance with data standards, clinical data with standards, reporting analytics, reporting functions, data warehousing operations, and compliance requirements.

In an embodiment, enterprise reference entity data model use cases span global health care EHR systems, such as Siemens MedSuite or Oracle Health EHR (formerly Cerner Millennium), for clinicals, financials, laboratory and laboratory quality control, and pharmacy operations, such as decision support systems and clinical conflict checking. Clinical documentation systems, such as Soarian Clinicals, MedSuite, and ISHMeds, integrate with reference enterprise data model through standardized interfaces.

In an embodiment, integration platforms facilitate connections between multiple proprietary EHR systems through integration architectures. Interoperability functions enable reconciliation between health information systems and EHR systems for problems, immunizations, and medications using HL7 and NCPDP workflows with ISO standards compliance. Electronic health record and financial systems connect with real-world data sources for pharmaceutical companies and regulatory agencies.

In an embodiment, a knowledge management system implements workflow processes for creating new integrated local dictionary structures. The knowledge management system selects new master dictionaries for medications, problems, laboratories, and related medical concepts, then creates and customizes local dictionaries tethered to master dictionary sources. Publication processes integrate local dictionaries to local systems while following maintenance workflows for integrated local dictionary management.

In an embodiment, the knowledge management system integrates existing local dictionary workflows by importing local dictionaries from EHR systems for medications, problems, laboratories, and related concepts. Tethering processes connect local dictionaries to corresponding master dictionary sources, followed by reconciliation of local items with tethered master dictionary content. Publication processes deliver integrated local dictionaries to local systems with subsequent maintenance workflow implementation.

In an embodiment, maintenance workflows for integrated local dictionaries involve re-importing local dictionaries with updates, reconciling updates with tethered master dictionary sources, and publishing integrated local dictionaries to an EHR system. Notification systems receive master dictionary updates and trigger appropriate reconciliation processes to maintain synchronization between local and master dictionary sources.

In an embodiment, AI workflow processes receive notifications from AI services regarding proposed classes for master dictionaries. A knowledge management system reviews proposed classes, reconciles updates with tethered master dictionary sources, and publishes updates to runtime systems. Notification systems enable automated proposal generation while maintaining human oversight for validation and approval processes.

In an embodiment, an ontology conversion system leverages content from existing ontology structures and converts ontological representations to logical formalism frameworks. Conversion processes address combinatorial explosion challenges and safety requirements while providing background knowledge for new axiom identification. Analysis encompasses atrial fibrillation care process models, standard atrial fibrillation protocols, condition management, immunization tracking, medication management, sepsis treatment protocols, and clinical decision support systems.

In an embodiment, a core concept model transforms ontology concepts from term-based representations to context-validated concepts. The core concept model establishes relationships beyond simple member relationships, incorporating hierarchical descriptions as ontological concepts. Concept structures include associated code sets with defined vocabularies, contexts, concept classes, and group memberships with subset relationships.

In an embodiment, the seamless exchange system addresses continuous client modifications to coded values without visibility into downstream impacts. Modified diagnosis codes potentially disrupt clinical decision support reminders or Fast Healthcare Interoperability Resources FHIR) mapping processes. Knowledge-based terminology service prototypes provide solutions for faster implementation through federated maintenance of concept relationships, elimination of knowledge asset silos, and reduced labor requirements for knowledge creation.

In an embodiment, a knowledge-based terminology service enables smart queries, meaningful data leverage with connections and representation, creation of computable artifacts, and galvanized decision support capabilities. Data quality improvements support patient care and research activities while reducing free text usage through better concept matching for codable concepts lacking coding or mapping relationships.

In an embodiment, a clinical decision support system infers and augments information within patient charts to supplement data not directly available in chart records. Semantic knowledge creation provides decision support through systematic analysis of patient medication lists, problem lists, allergies, and laboratory results.

In an embodiment, a clinical decision support system suggests alternative medications, optimizes treatment options, suggests differential diagnoses, validates clinical features of diagnoses, identifies treatment care gaps including opioid-related gaps, and translates clinical guidance and pathways into actionable clinical interventions for various conditions, such as diabetes mellitus and acute kidney failure. Immunization forecasting capabilities integrate with decision support functions.

In an embodiment, a digital assistant system for healthcare providers is a technology platform designed to streamline clinical workflows and administrative tasks through automation, voice recognition, and AI-powered tools. The digital assistant system helps healthcare professionals work more efficiently while maintaining focus on patient care. The digital assistant system for healthcare providers represents technological advancement in healthcare technology that enhances provider experiences and improves patient care outcomes. The digital assistant system focuses on connections between problems, diagnoses, medications, immunizations, procedures, and laboratory results to ensure providers access necessary information for clinical decision-making.

In an embodiment, the medication ontology establishes relationships between pharmaceutical classes, medications, conditions, contraindications, active ingredients, diagnoses, and anatomical structures. Medication ontology incorporates drug information from RxNorm, Multum, SNOMED CT, and ICD-10 sources with common side effects, post-marketing reported side effects, and contraindication relationships.

In an embodiment, the immunization ontology encompasses viral vaccines, drug components, contraindications, allergies, conditions, and severity factors. The immunization ontology processes patient age, gender, National Drug Code identifiers, Current Procedural Terminology codes, and ICD-10 diagnosis codes to determine vaccine administration appropriateness and contraindication identification.

In an embodiment, the immunization ontology evaluates specific vaccine scenarios, such as rotavirus and hepatitis B vaccine administration, for patients with severe combined immunodeficiency disease, vaccine component allergies, and previous vaccination history. Ontological relationships enable dynamic inference of contraindications based on patient-specific factors and vaccine composition data.

In an embodiment, the technical implementation framework incorporates runtime storage systems, smart query capabilities, reasoning engines for design and runtime operations, full-text search functionality, and numeric search with computed search capabilities.

In an embodiment, a concept categorization system enables knowledge architects and clients to add and integrate new terminology concepts, value sets, categories, and terminology systems. New variant identification encompasses emerging conditions, such as COVID variants, and experimental treatments through systematic evaluation processes. Code set standardization decisions span enterprise-wide implementations through comparative analysis of terminology definitions across organizational systems.

In an embodiment, the concept categorization system identifies unmatched codes, redundant codes, and orphaned codes across content management tools, forms, workflows, and external sources. Client code set integration drives standards updates through collaborative feedback mechanisms. Platform synchronization capabilities enable content integration across Event Set Hierarchy, Bedrock, Content Management Tools, and Ontology tools.

In an embodiment, development teams categorize concepts to enable smarter deduplication processes and improved ranking algorithms. Contextual data linking, retrieval, and display capabilities enhance concept understanding. Patient record curation functions support provider decision-making through recommendation engines and smart search capabilities.

In an embodiment, a provider management system addresses complex patient scenarios where conditions span multiple medical specialties. Nephrotic syndrome caused by diabetes type conditions appear on problem lists for nephrology and endocrinology specialists through shared diagnostic categorization. Competitive advantages emerge through interoperable platforms and knowledge-driven applications for managing complex patient populations.

In an embodiment, a seamless exchange reconciliation enables clinician users to view concepts grouped by parent classifications, including food allergies, beta blockers, and anticoagulants. Problem groupings encompass high cholesterol with hyperlipidemia, diabetes with diabetes mellitus, and related condition clustering. Patient summary document generation utilizes categorization functions for organized information presentation.

In an embodiment, a clinical guidance documentation system processes clinical pathways through LLM extraction and codification processes. Concept classification creates knowledge graphs from clinical documentation, while patient data extraction and codification generates subgraph structures for individual patients. Graph-based representations enable attribute application without complete modeling requirements.

In an embodiment, an LLM processing creates relationships, performs extraction operations, and interprets usage patterns from clinical guidance data. Machine learning methods target recommendation use cases, including missing vaccination identification, treatment optimization, and care gap analysis. Codification processes transform unstructured clinical guidance into computable formats suitable for automated decision support.

In an embodiment, clinical decision rules for immunization forecasting translate medical guidelines into logical statements. Hepatitis B recommended ranges encompass birth to four weeks for initial doses, four weeks to sixteen weeks for second doses, and six months to nineteen months for final doses. Special population considerations include preterm infants and infants born to hepatitis B surface antigen positive mothers.

In an embodiment, a diabetes mellitus management system integrates with chronic kidney disease risk assessment through standardized clinical guidelines. American Diabetes Association Standards of Care provide foundational frameworks for diabetes care, general treatment goals, and quality evaluation tools. Professional Practice Committee oversight ensures annual updates and evidence-grading systems for clinical practice recommendations.

In an embodiment, drug indication analysis connects Sotalol with SNOMED CT concepts through hasMultumIndication relationships. Subclass relationships link Sotalol classifications to broader pharmaceutical categories while maintaining specific indication mappings. Query capabilities enable traversal from drug concepts to condition hierarchies, side effect profiles, and contraindication relationships. A silver sulfadiazine drug example demonstrates side effect relationship modeling through hasMultumCommonSideEffect properties. Common side effects include leukopenia, application site rash, and pruritus conditions with corresponding SNOMED CT mappings. Query functionality retrieves drugs with specific side effect profiles and conditions treated by particular medications.

In an embodiment, a drug interaction visualization represents complex relationship networks between pharmaceutical substances and medical conditions through graph-based representations. Visual mapping displays connections between drug classifications, therapeutic indications, contraindications, and patient-specific factors. Interactive exploration enables clinicians to understand medication impacts across multiple physiological systems.

In an embodiment, medication ontology insights incorporate pharmaceutical classes, medication relationships, contraindication mappings, brand name associations, indication relationships, and anatomical structure connections. Side effect categorization encompasses common effects and post-marketing reported events with severity gradations and frequency classifications.

In an embodiment, medication relationship modeling connects RxNorm identifiers, Multum classifications, SNOMED CT concepts, and ICD-10 diagnostic codes through structured relationship frameworks. Drug therapeutic categories establish hierarchical classifications, while active ingredient mappings enable generic and brand name cross-referencing capabilities.

In an embodiment, an immunization ontology encompasses viral vaccine categories, drug component relationships, contraindication mappings, allergy associations, condition prerequisites, and severity reaction classifications. Patient scenarios incorporate age factors, gender considerations, National Drug Code identifiers, Current Procedural Terminology codes, and ICD-10 diagnosis codes for comprehensive vaccination decision support.

In an embodiment, a vaccine contraindication analysis processes patient conditions, including severe combined immunodeficiency disease, component allergies, and previous adverse reactions. Ontological reasoning enables dynamic contraindication identification based on patient-specific factors, vaccine composition data, and clinical guideline integration. Output generation provides contraindication explanations with supporting clinical rationale.

In an embodiment, a clinical data insights system provides semantic graph capabilities for healthcare provider queries about patient medication appropriateness, therapeutic alternatives, side effect profiles, contraindication identification, and care gap analysis. Query processing encompasses medication selection for specific conditions, adverse event identification, and treatment optimization recommendations.

In an embodiment, a medication appropriateness analysis determines patient suitability for beta blocker therapy, contraindications for vaccine administration, including MMR vaccines, travel vaccination requirements for specific geographic destinations, and alternative treatment options for patients with known allergies or contraindications.

In an embodiment, care gap identification processes encompass opioid-related treatment gaps, missing medication recommendations, duplicate medication detection, vaccination status verification, and condition-specific treatment requirements. Analysis capabilities extend to laboratory parameter monitoring, drug-laboratory interactions, and condition progression tracking.

In an embodiment, semantic reasoning capabilities support complex clinical queries, including medication ranking by likelihood of causing specific adverse effects, condition-medication interaction analysis, therapeutic alternative identification for contraindicated drugs, and comprehensive medication review for polypharmacy patients requiring multiple therapeutic interventions.

In an embodiment, an enterprise knowledge architecture addresses terminology concept integration, value set standardization, category establishment, and terminology system coordination across healthcare organizations. Variant identification processes encompass emerging medical conditions, experimental treatments, and evolving therapeutic approaches requiring systematic evaluation and integration.

In an embodiment, a knowledge asset management eliminates redundant terminologies, conflicting order sets, and inconsistent value set definitions through centralized governance processes. Labor reduction in knowledge creation relies on existing structural frameworks and client input mechanisms that streamline terminology development and maintenance workflows.

In an embodiment, an intelligent query system encompasses meaningful data connections, relationship discovery, and representation capabilities that support clinical decision-making processes. Computable artifact creation galvanizes decision support systems while improving data quality for patient care and research applications through standardized semantic frameworks.

### 6. Practical Applications, Advantages, & Improvements

The described system offers significant practical applications and advantages in the field of healthcare informatics. EHR systems experience substantial improvements through the integration of proprietary and standardized medical terminologies. The knowledge graph generation process enhances interoperability between diverse medical data sources, enabling more comprehensive patient care. Healthcare providers benefit from improved clinical decision support, as the system analyzes patient data against the rich knowledge graph to generate relevant suggestions. The technical improvement in the computer network manifests through enhanced data processing capabilities, allowing for real-time analysis of complex medical information. Efficiency in healthcare delivery increases as providers access more accurate and contextualized patient information. The system's ability to bridge proprietary and standardized vocabularies addresses a long-standing challenge in medical informatics, fostering better communication between different healthcare entities. Patient safety improves through more accurate medication management and comprehensive health assessments. The integration of machine learning models with the knowledge graph allows for continuous refinement of the system's analytical capabilities, ensuring adaptability to evolving medical knowledge and practices.

### 7. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

### 8. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

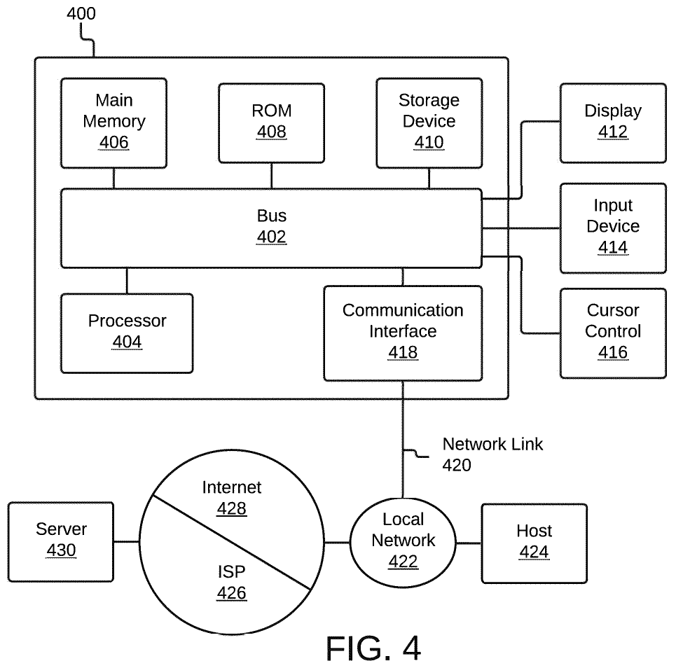

For example, FIG. 4 is a block diagram that illustrates a computer system 400 upon which an embodiment of the disclosure may be implemented. Computer system 400 includes a bus 402 or other communication mechanism for communicating information, and a hardware processor 404 coupled with bus 402 for processing information. Hardware processor 404 may be, for example, a general-purpose microprocessor.

Computer system 400 also includes a main memory 406, such as a random-access memory (RAM) or other dynamic storage device, coupled to bus 402 for storing information and instructions to be executed by processor 404. Main memory 406 also may be used for storing temporary variables or other intermediate information during execution of instructions to be executed by processor 404. Such instructions, when stored in non-transitory storage media accessible to processor 404, render computer system 400 into a special-purpose machine that is customized to perform the operations specified in the instructions.

Computer system 400 further includes a read only memory (ROM) 408 or other static storage device coupled to bus 402 for storing static information and instructions for processor 404. A storage device 410, such as a magnetic disk or optical disk, is provided and coupled to bus 402 for storing information and instructions.

Computer system 400 may be coupled via bus 402 to a display 412, such as a cathode ray tube (CRT), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane.

Computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor 404 executing one or more sequences of one or more instructions contained in main memory 406. Such instructions may be read into main memory 406 from another storage medium, such as storage device 410. Execution of the sequences of instructions contained in main memory 406 causes processor 404 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 410. Volatile media includes dynamic memory, such as main memory 406. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid-state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

Computer system 400 also includes a communication interface 418 coupled to bus 402. Communication interface 418 provides a two-way data communication coupling to a network link 420 that is connected to a local network 422. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN. Wireless links may also be implemented. In any such implementation, communication interface 418 sends and receives electrical, electromagnetic or optical signals that carry digital data streams representing various types of information.

Network link 420 typically provides data communication through one or more networks to other data devices. For example, network link 420 may provide a connection through local network 422 to a host computer 424 or to data equipment operated by an Internet Service Provider (ISP) 426. ISP 426 in turn provides data communication services through the worldwide packet data communication network now commonly referred to as the “Internet” 428. Local network 422 and Internet 428 both use electrical, electromagnetic or optical signals that carry digital data streams. The signals through the various networks and the signals on network link 420 and through communication interface 418, which carry the digital data to and from computer system 400, are example forms of transmission media.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution.

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

What is claimed is:

1. A non-transitory computer readable medium comprising instructions which, when executed by one or more hardware processors, causes performance of operations comprising:
processing proprietary terminology concepts from an electronic source to identify matching standardized medical terminology concepts from a standardized medical terminology source;
generating a knowledge graph incorporating the proprietary terminology concepts, the standardized medical terminology concepts and relationships between the proprietary terminology concepts and the standardized medical terminology;
receiving patient data including one or more of a problem list, a medication list, and lab results;
analyzing the patient data against the knowledge graph to generate a suggestion for an electronic health record (EHR) system; and
providing the suggestion to the EHR system for display.

2. The non-transitory computer readable medium of claim 1, wherein the operations further comprise:
processing natural language queries about a patient's medications and conditions using the knowledge graph to generate responses.

3. The non-transitory computer readable medium of claim 1, wherein creating the knowledge graph further comprises:
establishing relationships between medications and conditions by linking drug indications and side effects to corresponding diagnoses and problems within the knowledge graph.

4. The non-transitory computer readable medium of claim 1, wherein the operations further comprise:
extracting dosing information from the electronic source; and
using a large language model to interpret and reason about the extracted dosing information.

5. The non-transitory computer readable medium of claim 4, wherein the operations further comprise:
evaluating whether patients are on optimal medication doses for their conditions based on the interpreted dosing information.

6. The non-transitory computer readable medium of claim 1, wherein the operations further comprise:
receiving a query from the EHR system through a chatbot interface; and
analyzing the query against the knowledge graph to produce response through the EHR system.

7. The non-transitory computer readable medium of claim 6, wherein:
the EHR system receives auditory input through the chatbot interface; and
the auditory input is converted into the query.

8. The non-transitory computer readable medium of claim 1, wherein the standardized medical terminology source comprises the Systemized Nomenclature of Medicine Clinical Terms (SNOMED CT).

9. The non-transitory computer readable medium of claim 1, wherein the operations further comprise:
deduplicating the medication list using a semantic understanding of drug information from the knowledge graph.

10. The non-transitory computer readable medium of claim 1, wherein the operations further comprise:
generating automated summaries of patient records by extracting salient details about a patient's conditions, medications, and recent clinical events using the knowledge graph.

11. The non-transitory computer readable medium of claim 1, wherein processing proprietary terminology concepts uses natural language processing.

12. A method comprising:
processing proprietary terminology concepts from an electronic source to identify matching standardized medical terminology concepts from a standardized medical terminology source;
generating a knowledge graph incorporating the proprietary terminology concepts, the standardized medical terminology concepts and relationships between the proprietary terminology concepts and the standardized medical terminology;
receiving patient data including one or more of a problem list, a medication list, and lab results;
analyzing the patient data against the knowledge graph to generate a suggestion for an electronic health record (EHR) system; and
providing the suggestion to the EHR system for display, wherein the method is performed by at least one device including a hardware processor.

13. The method of claim 12, wherein the operations further comprise:
processing natural language queries about a patient's medications and conditions using the knowledge graph to generate responses.

14. The method of claim 12, wherein creating the knowledge graph further comprises:
establishing relationships between medications and conditions by linking drug indications and side effects to corresponding diagnoses and problems within the knowledge graph.

15. The method of claim 12, wherein the operations further comprise:
extracting dosing information from the electronic source; and
using a large language model to interpret and reason about the extracted dosing information.

16. The method of claim 15, wherein the operations further comprise:
evaluating whether patients are on optimal medication doses for their conditions based on the interpreted dosing information.

17. The method of claim 12, wherein the operations further comprise:
receiving a query from the EHR system through a chatbot interface; and
analyzing the query against the knowledge graph to produce response through the EHR system.

18. The method of claim 17, wherein:
the EHR system receives auditory input through the chatbot interface; and
the auditory input is converted into the query.

19. The method of claim 12, wherein the standardized medical terminology source comprises the Systemized Nomenclature of Medicine Clinical Terms (SNOMED CT).

20. A system comprising:
at least one device including a hardware processor;
the system being configured to perform operations comprising:
processing proprietary terminology concepts from an electronic source to identify matching standardized medical terminology concepts from a standardized medical terminology source;
generating a knowledge graph incorporating the proprietary terminology concepts, the standardized medical terminology concepts and relationships between the proprietary terminology concepts and the standardized medical terminology;
receiving patient data including one or more of a problem list, a medication list, and lab results;
analyzing the patient data against the knowledge graph to generate a suggestion for an electronic health record (EHR) system; and
providing the suggestion to the EHR system for display.
